(19) United States
(12) Patent Application Publication
(10) Pub. No.: US 2025/0288785 A1
(43) Pub. Date: Sep. 18, 2025

(54) SCENTED MEDICAL APPLICATORS

(71) Applicant: MJJ Group LLC, Port Washington, NY (US)

(72) Inventor: Martin Miller, Port Washington, NY (US)

(21) Appl. No.: 19/078,443

(22) Filed: Mar. 13, 2025

Related U.S. Application Data
(60) Provisional application No. 63/565,321, filed on Mar. 14, 2024.

(51) Int. Cl. A61M 35/00

(52) U.S. Cl. CPC A61M 35/006; A61M 2205/59

(57) ABSTRACT

Disclosed is a solution comprising an antiseptic agent and a fragrant solution in an amount to make the smell of the antiseptic agent, i.e. alcohol. The fragrant antiseptic solution can be infused into an applicator such as a pad. The applicator may be infused with the fragrant antiseptic solution, or a cover may contain the fragrant solution for later application to an antiseptic solution containing applicator. The fragrant and antiseptic solutions can be in the form of a fluid or a gel. In one embodiment there can be a foil lining surrounding the pad. In one embodiment there can be a scented paper surrounding the foil lining. In another embodiment there can be a cover surrounding the scented paper.

### REFERENCE TO PRIOR APPLICATION

This application claims the benefit of U.S. Provisional Application No. 63/565,321, filed Mar. 14, 2024, the entire contents of which are incorporated herein by reference.

### TECHNICAL FIELD

The present invention relates to a device and method for infusing a fragrance with an alcohol cleansing solution to be used in a disposable applicator.

### BACKGROUND OF THE INVENTION

Antiseptic pads/wipes provide a quick and easy antiseptic solution and have become a medical industry standard when preparing one's skin for an injection, amongst other procedures. Typically, these pads are pre-soaked in alcohol, commonly 2-proponol (isopropyl alcohol), packaged in individualized, disposable units and is effective in killing bacteria and viruses on the surface it is intended for. Although, while the use of alcohol is effective, some users may feel discomfort when presented with the smell of alcohol.

Currently, those who are sensitive to the smell of alcohol or other antiseptics, whether it be a strong aversion or a general discomfort, typically must bear through the odor when going into a medical office or when used at home. Further, individuals, for example children, may perceive the smell of alcohol as a negative stimulus, resulting in anxiety. There is no standardized or commonly available method to mask the odor of the alcohol. The solutions that are available focus on imparting a fragrant scent after use of the alcohol pad.

Therefore, there is a need for a medical pad containing an antiseptic solution that can be used by a user with a sensitivity to the odor of the antiseptic solution.

### SUMMARY OF THE INVENTION

Exemplary embodiments of the present invention may provide a solution, of which the solution can be prepared prior to infusion with a medical applicator or prepared by unsealing a package containing an alcohol infused medical applicator immediately prior to use, containing an antiseptic agent such as alcohol and a fragrance in an amount capable of masking the scent of the antiseptic agent. Other exemplary embodiments of the present invention may provide an alcohol infused medical applicator, and a fragrance in an amount capable of masking the scent of the antiseptic agent.

An exemplary embodiment further provides for a combination alcohol pad and fragrant solution that has effective disinfection capabilities while providing a user with a calming sensory experience.

At least one embodiment of the invention relates to a scented applicator such as a swab or pad which can either be self-contained in a package or when the swab or pad is released from a package it is then combined with a scent so that the scent can mask the smell of any anti-bacterial substance infused within the pad. The anti-bacterial substance in at least one embodiment can be alcohol.

Disclosed herein is a specialized scented/fragrant solution capable of masking the smell of an antiseptic solution, and methods of infusing a medical applicator comprising a pad with a scented solution. The formulation includes a carefully balanced concentration of medical-grade isopropyl alcohol, ensuring effective antiseptic properties, combined with a fragrance component that is either microencapsulated or directly infused to allow controlled scent release upon use. The pad can be infused with isopropyl alcohol for application on a person. A cover may also be provided containing a scented/fragrant solution. The scented solution can be in the form of a fluid or a gel. In one embodiment there can be a foil lining surrounding the pad and separating the pad from the scented solution. In one embodiment there can be a paper infused with the scented solution surrounding the foil lining. In another embodiment there can be a cover surrounding the scented or fragrant paper. For purposes of this application the terms scent and fragrant, fragrance, scent, and odor can be used interchangeably.

### BRIEF DESCRIPTION OF THE DRAWINGS

Other objects and features of the present invention will become apparent from the following detailed description considered in connection with the accompanying drawings which disclose at least one embodiment of the present invention. It should be understood, however, that the drawings are designed for the purpose of illustration only and not as a definition of the limits of the invention.

In the drawings, wherein similar reference characters denote similar elements throughout the several views:

FIG. 1 is a view of an applicator for use with a fragrant antiseptic solution according to an embodiment of the present invention;

FIG. 2 is a side view of the applicator of FIG. 1;

FIG. 3 is a view of the applicator of FIG. 1 within a packaging;

FIG. 4 is a side cross-sectional view of the applicator and packaging of FIG. 3;

FIG. 5 is a view of the applicator and packaging of FIG. 3 showing perforations disposed on the packaging;

FIG. 6 is a side view of the applicator of FIG. 1 with a scented permeable cover film;

FIG. 7 is a view of the applicator of FIG. 1 with an additional permeable polymer element which is infused with a scent;

FIG. 8 is a side view of the applicator of FIG. 1 with a permeable cover;

FIG. 9 is a side view of the applicator of FIG. 8 within packaging;

FIG. 10 is a side view of the applicator of FIG. 1 within packaging and having a foil;

FIG. 11 is another embodiment of an applicator showing multiple applicators in packaging;

FIG. 12 is a side view of another embodiment of an applicator having a foil lining and a scented paper film;

FIG. 13 shows another embodiment of a scented material alcohol infused pad combination;

FIG. 14 is a front view of another embodiment of an applicator in packaging and having a fragrant solution disposed on an outer scented paper-like material;

FIG. 15 is a side view of the applicator and packaging of FIG. 14;

FIG. 16 is a front view of the packing of FIG. 14 being torn open;

FIG. 17 is a front view of another embodiment of an applicator in packaging;

FIG. 18 is a side view of the applicator and packaging of FIG. 17;

FIG. 19 shows a process for creating the scented material and alcohol infused pad in a single package.

### DETAILED DESCRIPTION

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure.

Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left, right, are for illustrative purposes only and can be varied within the scope of the disclosure.

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Reference will now be made in detail to the exemplary embodiments of the present disclosure, which are illustrated in the accompanying tables and figures.

Disclosed herein is an antiseptic solution having a scent mixed with the solution for masking the unpleasant odor associated with the antiseptic. Preferably, the antiseptic is an alcohol, such as isopropyl alcohol (isopropanol), but other antiseptic solutions, such as ethyl alcohol, are contemplated. It is contemplated that the scented antiseptic solution may be in a liquid or gel form, and preferably a synthetic polymer medical pad carries and administers the solution.

The fragrant solution comprises of a scent having a weight percent composition of a range of 0.1-0.5% weight for masking the smell of the antiseptic to an average user. In a preferred embodiment, the combination fragrant antiseptic solution comprises of at least 70% isopropanol, at least 0.1% concentrated fragrant solution, and a solvent such as water, which accounts for up to 29.9% weight of the total solution. It is contemplated that variations to the mixture may be made to account for intensity of the scent of the fragrant solution being used as well as the needs of the user, discussed infra. Thus, it is contemplated that the concentrated fragrant solution may range from 0.1% to 0.5%, preferably approximately 0.3%, weight of the total solution, ideally minimally diluting the water solvent in order to maintain a preferred evaporation rate and protein denaturation of microbes as that of a standard 70% isopropyl alcohol solution. It is preferred that 70% weight isopropanol (or other contemplated antiseptic solutions) remain the same in other embodiments of the solution for its optimal evaporation rate on human skin as opposed to higher or lower purity antiseptic solutions. While ranges are depicted, said ranges are merely exemplary and more or less fragrant solution may be used depending on the concentration of the specific fragrant solution being used. It is further contemplated that the fragrant antiseptic solution may be provided in the form of a gel by known methods such as introducing a gelling agent and/or an emollient or by other known methods in the art.

The specific scent applied to the fragrant solution can vary in amount and intensity depending on the scent used and the needs of an individual. For example, it would be ideal to formulate a fragrant antiseptic solution for a child that has a less potent fragrant solution having a scent of bubblegum or grape, and a more potent fragrant solution for an adult with a higher aversion to the smell of alcohol having a scent of lavender or vanilla. Alternatively, it is ideal that an embodiment would be applicable to all individuals, including those who are not aversive to the smell of alcohol, with scents that are generally found pleasant to many individuals, such as vanilla, lavender, and strawberry. The specific scents disclosed herein are merely exemplary, and any of the scents disclosed can be used with any level of potency of the fragrant solution and other scents are contemplated.

The fragrant antiseptic solution is preferably administered topically by way of a carrier component/applicator, such as an absorbent fibrous polymer-type pad. As used herein, carrier component or applicator shall refer to a material capable of being infused or can otherwise transport both the antiseptic solution and fragrant solution either separately or together. While polymer pads are preferred, other materials known for delivering antiseptic solutions, such as cotton, are contemplated. Preferably the pad material is a highly absorbent non-woven, or cotton material fabric which can ensure uniform distribution of the solution while preventing premature evaporation of both alcohol and fragrance. During storage and transportation, it is preferred, but not required, to keep the antiseptic solution and the fragrant solution separated prior to use.

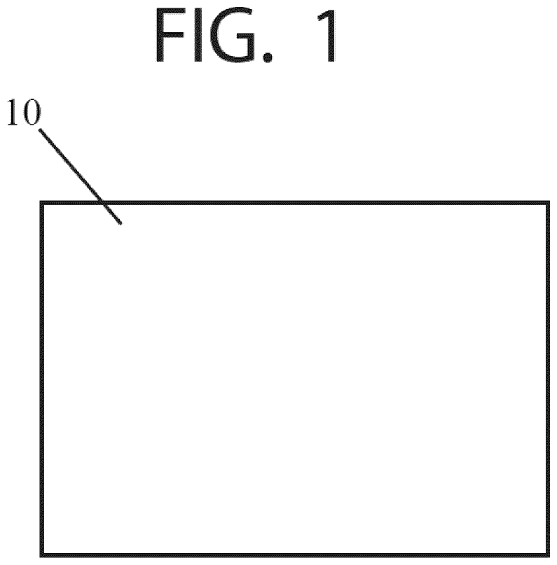

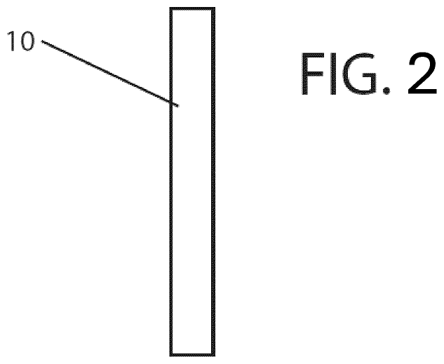

FIG. 1-2 is a view of an applicator 10 containing an antiseptic solution, which can either be infused with a fragrant solution or configured to be infused with a fragrant solution.

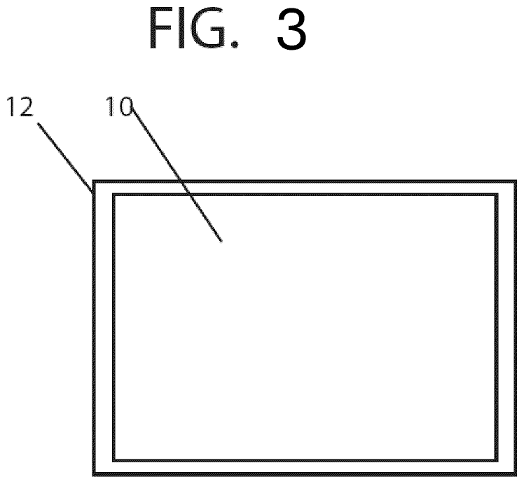

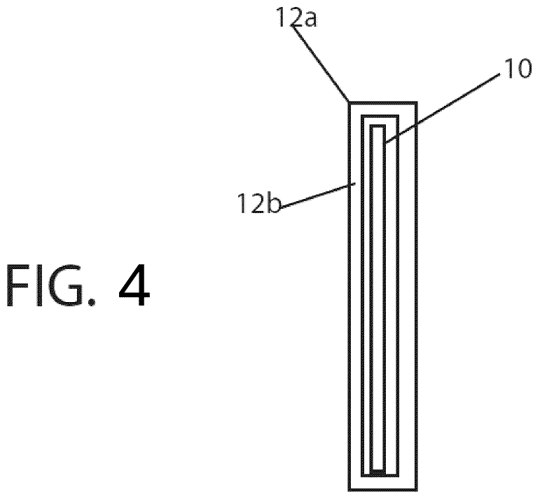

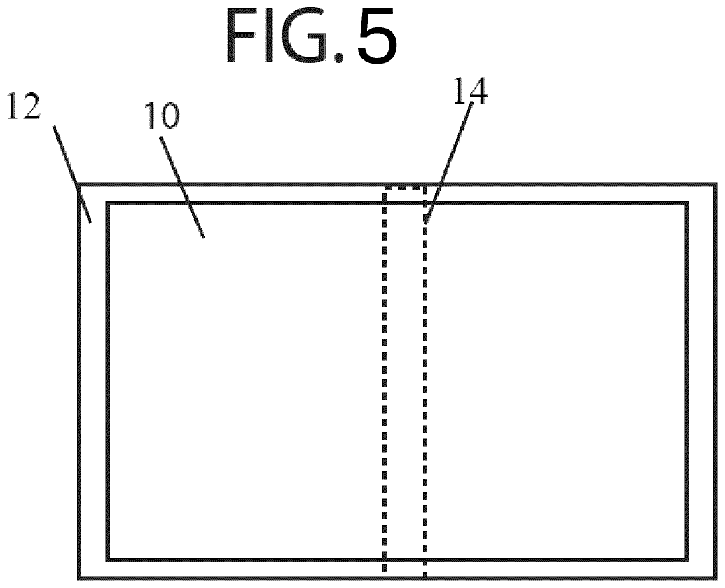

FIGS. 3-4 are views of an applicator 10 within an enclosure 12 according to an embodiment of the present disclosure. As seen in FIG. 4, the packaging may include a solid impermeable layer 12a on the outside and a permeable layer 12b on the inside of the packaging 12, of which the fragrant solution is infused within the permeable layer 12b. In this embodiment, the antiseptic solution disposed on applicator 10, and the fragrant solution infused in permeable layer 12b are encapsulated within packaging 12 during shipping and storage. As seen in FIG. 5 it is contemplated that packaging 12 may also contain a perforated section 14 centrally disposed for obtaining applicator 10 from within packaging 12.

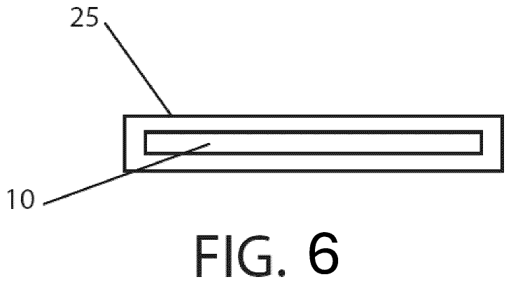

In another embodiment as seen in FIG. 6, an outer layer comprising a scented permeable cover film 25 is provided which can be infused with the scented solution such that when the cover film 25 is torn away from the applicator 10, the scented material is released from the cover film 25 and infused into the applicator 10. It is contemplated that the cover film 25 of the present embodiment can be used in lieu of the permeable layer 12b of FIG. 4 when stored in an enclosure such as packaging 12.

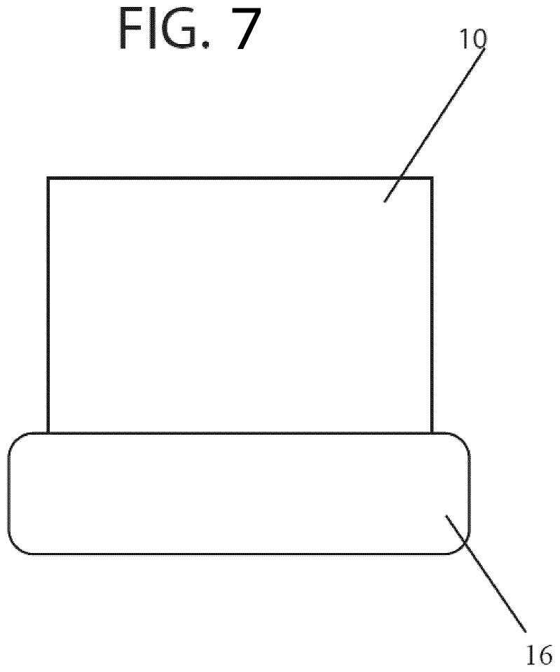

In a further embodiment, FIG. 7 is a view of applicator 10 according to an embodiment of the invention in which an additional permeable polymer element 16 infused with a scented solution is provided. It is contemplated that the polymer permeable element 16 can be in the form of a permeable polymer element such as a sponge, configured to hold a fragrant solution therein. When the permeable polymer element is compressed, it allows for the fragrant solution to transfer into the antiseptic solution infused applicator 10 such that the applicator 10 itself becomes scented. It is contemplated that the permeable polymer element 16 of the present embodiment can be used in lieu of the permeable layer 12b of FIG. 4 when stored in an enclosure such as packaging 12.

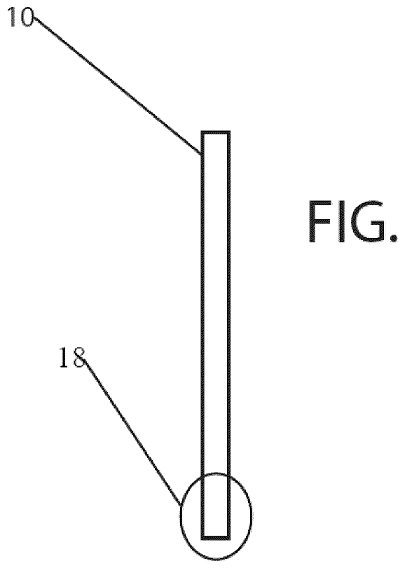

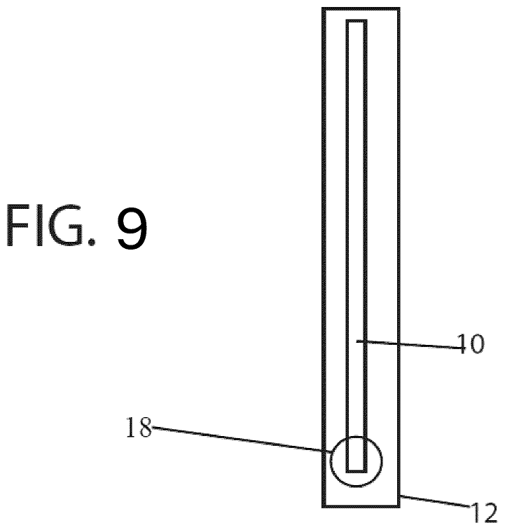

FIG. 8 is a side view of applicator 10 according to an alternative embodiment having a permeable base 18 disposed at an end of the applicator 10. The permeable base 18 can be infused with fragrant solution so that when depressed, the base 18 allows for the leakage of the fragrant solution onto the applicator 10. It is contemplated that the permeable base 18 of the present embodiment can be used in lieu of the permeable layer 12b of FIG. 4 when stored in an enclosure such as packaging 12, as seen in FIG. 9.

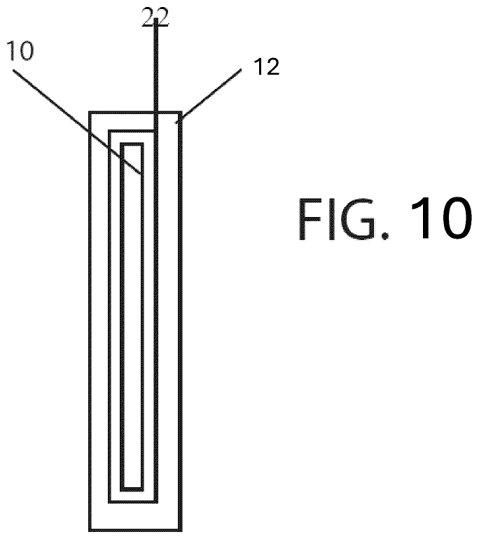

In a further embodiment, as seen in FIG. 10, applicator 10 is disposed within packaging 12 and having a foil 22. For example, applicator 10 is disposed inside of a foil material 22, the fragrant solution is disposed between the inner layer of foil material 22 and the outer packaging 12. Thus, when the packaging and the foil material are broken and/or torn the fragrant solution can then infuse into the applicator 10. It is contemplated that at least two applicators 30 surrounded by a foil 26 and an outer cover 12 may be joined by a perforated fold 24 disposed on adjoining edges of the outer cover 12, as seen in FIG. 11. When the at least two outer covers 12 are torn apart along the perforated fold 24, an opening is provided which allows the applicators 10 to be removed. Upon tearing of foil 26, the fragrant solution disposed between outer cover 12 and the inner foil 26 can be infused with antiseptic solution containing applicator 30.

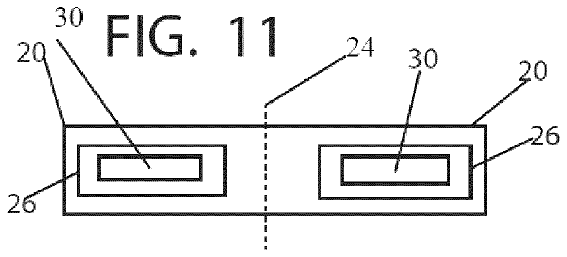

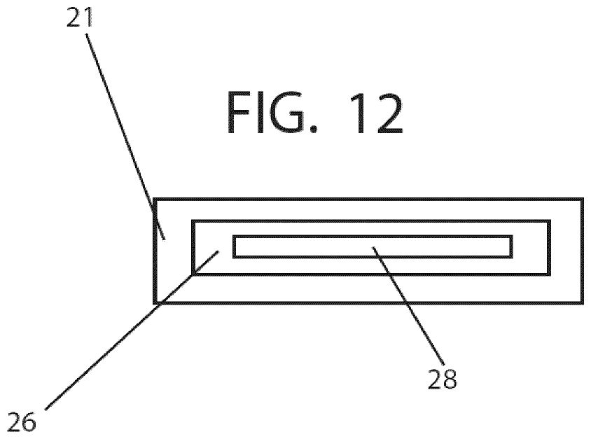

In a further embodiment as seen in FIG. 12, an applicator 28 is provided having a foil lining 26 and a paper film 21 infused with the fragrant solution. The scented paper film 21 can be wiped against applicator 28 to infuse the applicator 28 with the fragrant solution.

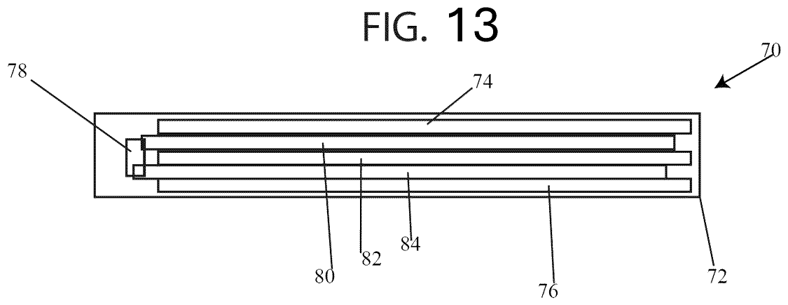

In a further embodiment as seen in FIG. 13 packaging 72 surrounds scented or fragrant material 74 and 76 of which scented or fragrant material 74 and 76 surround applicator 82. It is contemplated that the fragrant material can be a permeably polymer element such as a sponge. Separating the scented or fragrant material from an internal applicator 82 are separation layers 80 and 84 coupled at a common edge by a binder 78. In a preferred further embodiment, the separation layers are formed form foil although other non-permeable separating layers are contemplated. When the packaging 72 is opened and the binder 78 coupled to the separation layers 80 and 84 are removed, the applicator 82 comes in contact with the scented material or layers 74 and 76, infusing the fragrant solution into the applicator 82 on both sides.

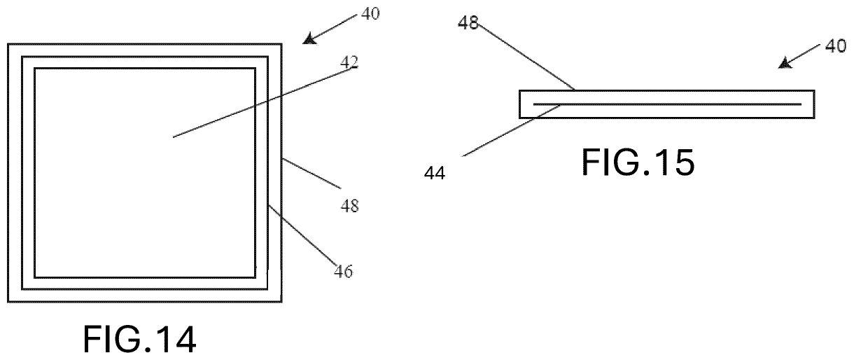

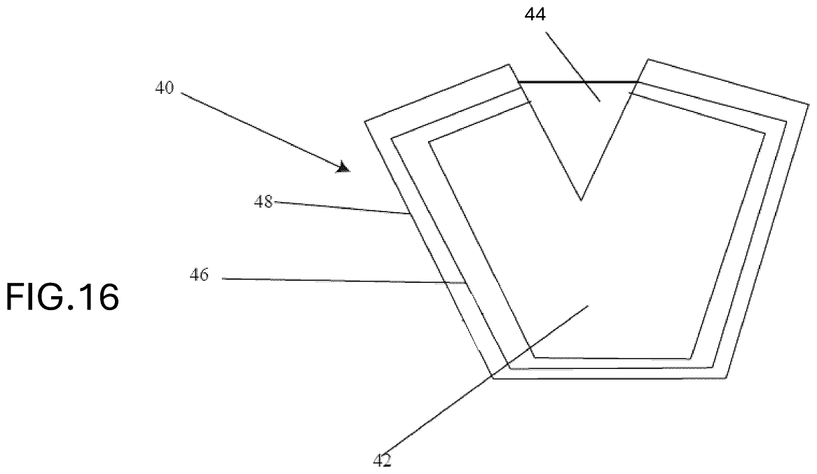

FIG. 14 is a front view of packaging 40 for an applicator (not shown) having the antiseptic solution infused therein according to another embodiment wherein the packaging 40 includes an outer seal 48, an exterior package section or seal 46 and a material for holding a scent 42 infused with the fragrant solution. It is contemplated that the material for holding a scent 42 is disposed externally on the package 40 and is a scented paper-like material. FIG. 15 shows a side view of the exterior outer seal 48 with the interior applicator 44 shown therein. FIG. 16 is a view of a packaging 40 for the applicator 44 of FIG. 14 torn open. This packaging 40 has outer seal 48 torn open. In addition, seal 46 is also torn open thereby allowing the fragrant solution disposed on scented paper-like material 42 to permeate into the air. This allows for the scented solution, in a vapor form, to scent the surrounding environment and reduce the smell of the antiseptic solution disposed on applicator 44.

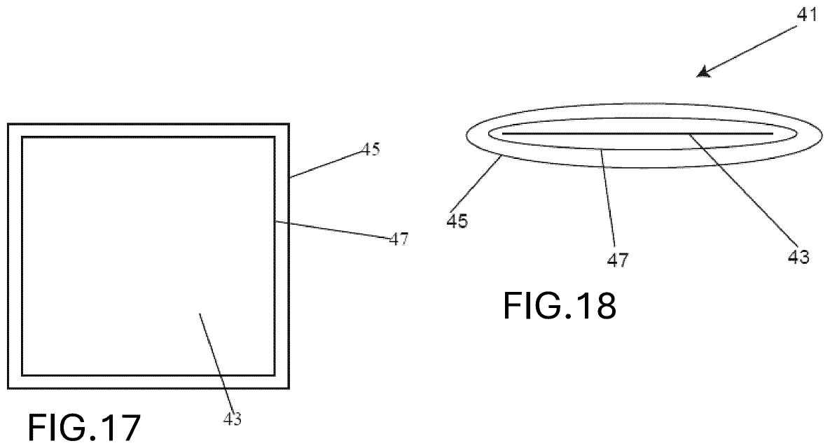

FIG. 17 is a front view of an applicator 43 according to another embodiment having an outer section or exterior packaging 47 infused with a fragrant solution with a sealed end section or outer seal 45. FIG. 18 is a side view of applicator 43 of FIG. 17 which shows applicator 43 being surrounded by exterior packaging 47 and an outer seal 45.

In all, there is disclosed multiple different embodiments which allow for the selective infusion of a fragrant solution into an applicator having an antiseptic solution already disposed therein. Other methods of infusing an antiseptic applicator, such as a pad or wipe, with a fragrant solution immediately prior to application on the skin or a surface are contemplated.

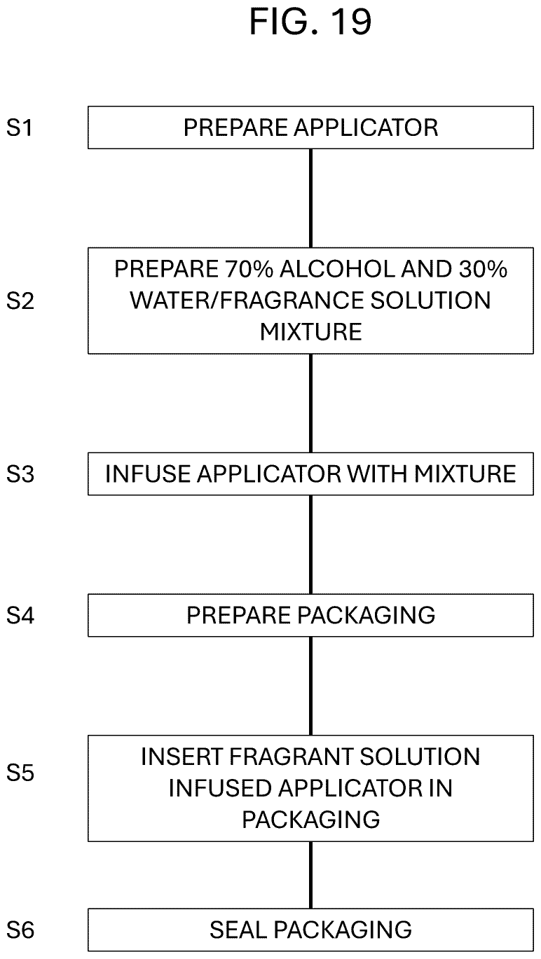

FIG. 19 is a process for packaging an embodiment of the applicator for transportation and storage. The process starts in step S1 wherein the applicator can be prepared. The applicator itself can be in the form of a gauze pad or other type of cotton or medical pad. Next, in step S2, the antiseptic fragrant solution is prepared, comprising 70% alcohol and 30% water and fragrant solution mixture. Next, in step S3 can be infused with the antiseptic fragrant solution. Next in step S4 the packaging can be prepared. In a preferred embodiment, the packaging comprises a non-permeable paper like polymer, such as polyethylene, although other materials that are non-permeable and can be easily torn are contemplated. It is further contemplated that the interior of the packaging can comprise laminated aluminum foil. Next, in step S5, the alcohol and fragrant solution infused applicator can be inserted into the packaging. Next, in step S6 the packaging can be sealed. It is contemplated that the steps described can be adapted and changed to suit the antiseptic fragrant solution delivery embodiments described above.

Accordingly, it is to be understood that other methods of packaging and transporting the fragrant antiseptic applicator may be employed, and the embodiments described herein are merely contemplated. Other methods known in the art of transporting applicators are also contemplated.

Accordingly, while at least one embodiment of the present invention have been shown and described, it is to be understood that many changes and modifications may be made thereunto without departing from the spirit and scope of the invention as defined in the appended claims.

What is claimed is:

1. A fragrant antiseptic solution for masking the odor of the antiseptic agent, comprising:
an antiseptic agent;
a fragrant solution,
wherein the ratio of the fragrant solution to the antiseptic agent is in an amount such that an odor emanating from the fragrant solution is capable of masking the odor emanating from the antiseptic agent.

2. The fragrant antiseptic solution of claim 1, wherein the antiseptic agent is an alcohol.

3. The fragrant antiseptic solution of claim 2, wherein the antiseptic agent is isopropanol.

4. The fragrant antiseptic solution of claim 3, wherein the solution further comprises at least 70% isopropyl alcohol, a range of approximately 0.1% to approximately 0.5% fragrant solution, and water.

5. The fragrant antiseptic solution of claim 4, wherein the further solution comprises at 70% isopropyl alcohol, approximately 0.3% fragrant solution, and water.

6. The fragrant antiseptic solution of claim 1, wherein the fragrant solution is scented with a strawberry, vanilla, lavender, bubble gum or grape odor.

7. An applicator having the fragrant antiseptic solution of claim 1 infused therein.

8. The applicator of claim 7, wherein the applicator is a fibrous polymer pad.

9. The applicator of claim 8, wherein the applicator is cotton.

10. An applicator, comprising:
a carrier component configured to be infused with a solution;
the solution comprising an alcohol and a fragrant solution infused into the carrier component wherein the fragrant solution is configured to mask the smell of alcohol in the carrier component.

11. The applicator of claim 10, wherein the solution comprises of a scent having a weight percent composition range of approximately 0.1% to approximately 0.5% weight in a solution comprising 70% isopropyl alcohol and water for masking the smell of isopropyl alcohol to an average user.

12. The applicator as in claim 10, further comprising a cover, wherein the solution is in the form of a liquid.

13. The applicator as in claim 10, wherein the solution is in the form of a gel.

14. The applicator as in claim 10, further comprising a cover wherein the cover has a plurality of perforations.

15. The applicator as in claim 14, further comprising a foil lining wherein the foil lining is positioned between the cover and the applicator.

16. The applicator as in claim 10, further comprising a scented paper in the form of a film, wherein the film is positioned around the applicator.

17. The applicator as in claim 10 further comprising a permeable cover covering a portion of the applicator.

18. The applicator as in claim 16, wherein the permeable cover is in the form of a permeable polymer element.

19. An applicator comprising:
a carrier component capable of carrying an antiseptic;
a foil lining surrounding said carrier component;
a scented paper surrounding said foil lining;
a cover surrounding said scented paper.

20. The applicator as in claim 19, wherein the scented paper is infused with a scented solution.
